(19) United States
(12) Patent Application Publication
(10) Pub. No.: US 2014/0249304 A1
(43) Pub. Date: Sep. 4, 2014

(54) LONG INTERFERING DSRNA SIMULTANEOUSLY INDUCING AN IMMUNE REACTION AND THE INHIBITION OF THE EXPRESSION OF TARGET GENES

(71) Applicant: RESEARCH & BUSINESS FOUNDATION SUNGKYUNKWAN UNIVERSITY, Suwon-si (KR)

(72) Inventors: Dong Ki Lee, Seoul (KR); Chan Il Chang, Daejeon (KR)

(73) Assignee: RESEARCH & BUSINESS FOUNDATION SUNGKYUNKWAN UNIVERSITY, Suwon-si (KR)

(21) Appl. No.: 14/123,056

(22) PCT Filed: May 30, 2012
(86) PCT No.: PCT/KR2012/004259

§ 371 Date: Apr. 24, 2014

(30) Foreign Application Priority Data
May 30, 2011 (KR) 10-2011-0051641

(51) Int. Cl. C12N 15/113

(52) U.S. Cl. CPC C12N 15/113
USPC 536/24.5

(57) ABSTRACT

A long interfering dsRNA (liRNA) capable of inhibiting specific RNAi-mediated expression of target genes and promoting an immune reaction, and use thereof are provided. The long interfering dsRNA can be useful in inhibiting specific expression of target genes through an RNA-interfering reaction in a sequence-specific manner and inducing expression of interferon-β by stimulating a protein kinase R (PKR) path in a structure-dependent manner.

### CROSS-REFERENCE TO RELATED APPLICATION

This application claims priority to and the benefit of Korean Patent Application No. 2011-0051641, filed on May 30, 2011, the disclosure of which is incorporated herein by reference in its entirety.

### BACKGROUND

1. Field of the Invention

The present invention relates to a long interfering dsRNA capable of inhibiting expression of target genes and simultaneously promoting an immune reaction, and more particularly, to a long interfering dsRNA structure capable of inhibiting specific expression of target genes in a sequence-specific manner and inducing an immune reaction in a structure-dependent manner.

2. Discussion of Related Art

A long double-stranded RNA (dsRNA) sequence is mainly formed during viral replication, but is not present in eukaryotic cells. Therefore, eukaryotic organisms recognize long dsRNAs in a virus-associated molecule pattern, and cause a potent antiviral immune reaction. When the long dsRNAs are introduced into mammalian cells, protein kinase R (PKR) and 2,5-oligoadenylate synthetase (OAS) are activated (GANTIER, M. P. and WILLIAMS, B. R., Cytokine Growth Factor Rev., 18:363-371, 2007). The activated PKR phosphorylates a eukaryotic translation initiation factor, eIF-2α, to block translation initiation, and phosphorylates IκBα to activate an NF-κB path (GIL, J et al., Mol. Cell. Biol., 19:4653-4663, 1999). As a result, the activated PKR serves to cause cell death and increase expression of type-I interferon, for example, interferon-β. In turn, The OAS activated by the dsRNA activates RNase L to cause non-specific mRNA degradation and cell death (Iordanov et al., Mol. Cell. Biol., 21:61-72, 2001). Therefore, introduction of the long dsRNA into the mammalian cells results in potent anti-proliferative activities together with induction of various cytokines.

In addition, the antiproliferative activities and immunostimulatory activities of long dsRNAs such as polyinosinic:polycytidylic acid [poly(I:C)] have been effectively used to develop a new strategy for killing cancer cells. However, the poly(I:C) strongly and continuously expresses cytokines, and potentially causes cytotoxicity when the expression of the cytokines is not controlled.

Another current strategy of developing an RNA-based anticancer drug is based on an RNA interference (RNAi) mechanism. RNAi is a mechanism of inhibiting post-transcriptional expression of genes conserved in various species (HANNON, G. J., Nature, 418:244-251, 2002). When long dsRNAs are introduced into cells, they are digested into short dsRNAs of 21 to 23 bps in length by an RNase III-like enzyme referred to as “Dicer.” The short dsRNAs are recognized by an RNA-induced silencing complex (RISC), and an RNA strand having the thermodynamically unstable 5′-terminus is preferentially integrated into the active RISC complex to specifically digest a target mRNA. RNAi-based gene silencing has a significant potential as an anticancer drug since it has a potential of specifically inhibiting almost all oncogenes, including genes which are not targetable by small molecules or monoclonal antibodies (PECOT, C. V et al., Nat Rev Cancer, 11:59-67, 2010).

Long (0.3 to 1 kb) dsRNAs found originally in C. elegans have been successfully used to induce inhibition of sequence-specific gene expression in a wide range of organisms (Fire et al., Nature, 391:806-811, 1998). However, inhibition of RNAi-mediated specific gene expression using the long dsRNAs failed in mammalian cells. This is because non-specific mRNA degradation occurs due to an antiviral reaction caused by the long dsRNAs, and protein synthesis is inhibited (Stark et al., Annu. Rev. Biochem., 67:227-264, 1998).

Also, inhibition of specific gene expression in mammalian cells is induced using a synthetic RNA duplex of 19 bps having 3′ overhangs having an induction structure, which mimics a structure of a Dicer-cleaved product (Elbashir et al., Nature, 411:494-498, 2001). In this case, a small interfering RNA (Hereinafter referred to as “siRNA”) structure induced inhibition of specific gene expression without inducing interferon in mammalian cells and down-regulating non-specific mRNAs. For this reason, long RNA duplexes have been avoided as an RNAi-induced structure for most of studies in mammalian cells.

To develop an RNAi therapeutic agent, researchers have focused on induction of inhibition of specific gene expression without inducing an innate immune reaction. However, inhibition of siRNA-mediated gene expression together with immunostimulation can be effectively used for therapeutic use to develop an anticancer drug or an antiviral therapeutic agent (Schlee et al., Mol Ther, 14:463-470, 2006).

Accordingly, the present inventors made an ardent effort to provide a long dsRNA structure (hereinafter referred to as a “long interfering dsRNA” or “liRNA”) having nicks as a novel immunostimulatory RNAi-induced structure, designed a liRNA including siRNA units in which a plurality of base pairs are formed between overhangs, and found that the liRNA had innate functions of siRNA to specifically inhibit expression of target genes, and also had promoted an immune reaction. Therefore, the present invention was completed based on these facts.

### SUMMARY OF THE INVENTION

The present invention is directed to a long interfering dsRNA (liRNA) structure capable of inhibiting specific expression of target genes with siRNAs and simultaneously promoting an immune reaction.

According to an aspect of the present invention, there is provided a long interfering dsRNA (liRNA) to which a double-stranded siRNA having overhangs is linearly bound by means of complementary base-pair binding. Here, the double-stranded siRNA having the overhangs is composed of an antisense strand and a sense strand, each of which is 19 to 59 nucleotides (nts) in length, wherein the antisense strand and the sense strand are ligated to form a 13 to 50 bp complementary double helix structure, and the double-stranded siRNA has the overhangs of 4 to 46 nts in length bound to both the 5′ termini or both the 3′ termini of the double helix structure.

According to another aspect of the present invention, there is provided a composition for inhibiting expression of genes or promoting an immune reaction, which includes the liRNA.

According to still another aspect of the present invention, there is provided an antiviral composition including the liRNA.

According to yet another aspect of the present invention, there is provided an anticancer composition including the liRNA.

### BRIEF DESCRIPTION OF THE DRAWINGS

The above and other objects, features, and advantages of the present invention will become more apparent to those of ordinary skill in the art by describing in detail exemplary embodiments thereof with reference to the accompanying drawings, in which:

FIG. 1 is a diagram showing structures of liRNAs, that is, structures of liRNAs targeting Survivin or GFP;

FIG. 2 is a diagram showing a size distribution pattern of the liRNAs for comparison between poly(I:C) and siRNAs;

FIG. 3 is a graph illustrating the activities of the liRNAs targeting Survivin mRNA to inhibit gene expression. All data in the graph are represented by an average value±standard deviation of experiments carried out in triplicate, and a concentration of the liRNAs is indicated as a concentration of an antisense strand. FIG. 3A shows expression levels of GAPDH mRNA in liRNA-transfected cells. Here, the Y axis represents a level of GAPDH mRNA measured using the same amount of total RNAs from the liRNA-transfected samples. FIG. 3B shows expression levels of Survivin mRNA in the liRNA-transfected cells. Here, the Y axis represents a level of Survivin mRNA measured using the same amount of total RNAs from the liRNA-transfected samples. FIG. 3C shows the values obtained by dividing the level of Survivin mRNA by the level of GAPDH (control) mRNA;

FIG. 4 is an experimental graph illustrating induction of interferon caused by the liRNAs. HeLa cells were transfected with each of liRNAs (0.3 nM), and after 12 hours and 24 hours, an IFN-β level was measure using qRT-PCR. An IFN-β mRNA level of a mock-treated sample (0 nM) was set as 1. All the data in the graph is represented by an average value±standard deviation of experiments carried out in triplicate;

FIG. 5 is an experimental graph illustrating inhibition of growth of cancer cells by the liRNAs. HeLa cells were transfected with each of liRNA, siRNA, and poly(I:C), and then counted at a given point of time to determine cell growth. All the data in the graph is represented by an average value±standard deviation of experiments carried out in triplicate;

FIG. 6 is an experimental graph illustrating whether liRNA-mediated cell death is regulated in a PKR-dependent manner. All the data in the graph is represented by an average value±standard deviation of experiments carried out in triplicate; and

FIG. 7 is a diagram showing a structure of a liRNA targeting Survivin and β-catenin, and a structure of a liRNA in which siRNAs targeting the Survivin and the β-catenin are ligated by means of a linker.

### DETAILED DESCRIPTION OF EXEMPLARY EMBODIMENTS

Exemplary embodiments of the present invention will be described in detail below with reference to the accompanying drawings. While the present invention is shown and described in connection with exemplary embodiments thereof, it will be apparent to those skilled in the art that various modifications can be made without departing from the scope of the invention.

Unless specifically stated otherwise, all the technical and scientific terms used in this specification have the same meanings as what are generally understood by a person skilled in the related art to which the present invention belongs. In general, the nomenclatures used in this specification and the experimental methods described below are widely known and generally used in the related art.

The definition of the main terms used in the detailed description of the present invention is as described below.

The term “siRNA (small interfering RNA)” used herein refers to a short double-stranded RNA (dsRNA) mediating efficient gene silencing in a sequence-specific manner, that is, a small RNA fragment which is 19 to 23 nucleotides in length and produced by digesting a double-stranded RNA with a Dicer enzyme.

The term “target gene” used herein refers to a gene whose expression is selectively inhibited or inactivated by siRNA. Such inactivation is achieved by digesting mRNA of the target gene with the siRNA. In a preferred exemplary embodiment of the siRNA according to the present invention, an siRNA capable of forming a complementary bond with mRNA of Survivin and an siRNA capable of forming a complementary bond with mRNA of β-catenin have been used to inhibit expression of Survivin commonly expressed in most of the tumor cells. However, other tumor- or cancer-associated genes such as RAS, MYC, ERBB, BCR-ABL, TEL-AML1, BCL-22, and the like, and other disease-associated genes may also become target genes of the liRNA according to the present invention. When considering the guidance provided in this specification, a person having ordinary skill in the related art will recognize that other siRNA sequence-based liRNA molecules serving to reduce expression of any various target genes may be easily produced according to the methods widely known in the related art.

The term “liRNA” used herein refers to a long double-stranded RNA in which units composed of siRNAs in which a plurality of base pairs are formed between overhangs are repeated, that is, a long double-stranded RNA capable of inhibiting specific expression of target genes in a sequence-dependent manner and simultaneously inducing an immune reaction in a structure-dependent manner. The number of the siRNAs constituting the liRNA is not limited, and is intended to encompass all the liRNAs in which two or more, that is, three, or four or more different kinds of siRNAs are repeated.

In one aspect of the present invention, a long interfering dsRNA (liRNA) to which a double-stranded siRNA is linearly ligated by means of complementary base-pair binding is provided. Here, the double-stranded siRNA is composed of an antisense strand and a sense strand, each of which is 19 to 59 nts in length, wherein the antisense strand and the sense strand are ligated to form a 13 to 50 bp complementary double helix structure, and the double-stranded siRNA has overhangs of 4 to 46 nts in length bound to both the 5′ termini or both the 3′ termini of the double helix structure.

According to the present invention, the overhangs of both the termini of the double helix structure may be characterized in that they have sequences complementary to each other. Here, the overhangs may be present in both the 5′ termini or both the 3′ termini of the double helix structure. The overhangs of the siRNA sequence may be rapidly linearly ligated at some length through complementary base-pair binding due to the presence of such complementary sequences. Also, the overhangs may be characterized in that they have a melting temperature (Tₘ) of greater than 30° C. When the Tₘ of the overhangs is less than or equal to 30° C., the complementary sequences may be unwound at an in vivo temperature without maintaining a duplex.

In the present invention, the antisense strand complementary to the sense strand may be characterized in that it has a sequence having a homology of at least 70% with respect to an mRNA sequence of a target gene. In this case, the overhangs may have sequences which may be or may not be complementary to the mRNA sequence of the target gene.

In the present invention, the overhang of the antisense strand of the liRNA may be characterized in that it has a sequence having a homology of at least 70% with respect to the mRNA sequence of the target gene.

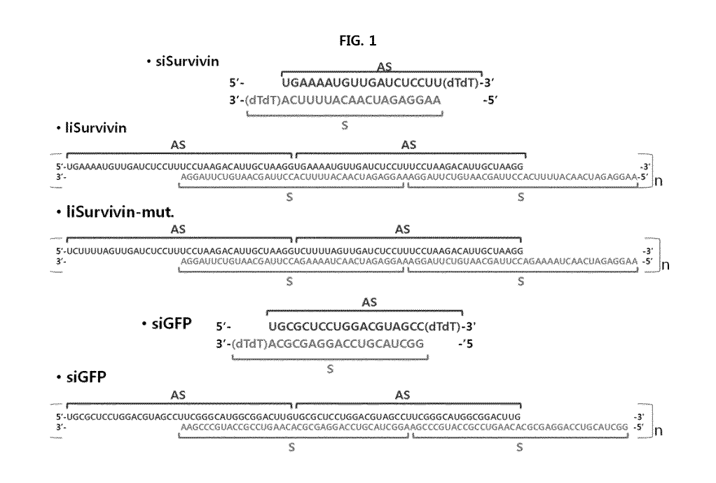

According to one exemplary embodiment of the present invention, it was confirmed that a liRNA in which an antisense strand has a sequence complementary to an siSurvivin sequence and an mRNA sequence of Survivin was constructed to target cancer-associated genes (FIG. 1), an expression level of Survivin mRNA was inhibited, and interferon was induced, thereby significantly inhibiting growth of cancer cells.

The siRNAs constituting the liRNA according to the present invention functions to inhibit gene expression in a sequence-specific manner, and induction of the immune reaction is due from a structure of the liRNA. Therefore, the siRNAs may be replaced with any siRNA sequences targeting genes associated with other diseases such as viral diseases other than cancer.

According to another aspect of the present invention, the antisense strand of the liRNA may be characterized in that it has a sequence having a homology of at least 70% with respect to mRNA sequences of two or more different target genes. The liRNA in which units composed of siRNAs targeting the different target genes are repeated may effectively inhibit expression of two or more genes at the same time.

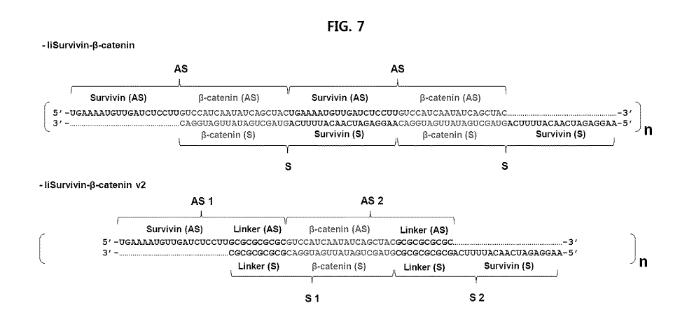

According to one exemplary embodiment of the present invention, a liRNA in which units composed of siSurvivin and siβ-catenin are repeated was designed to target the cancer-associated genes (FIG. 7). However, it will be apparent to those skilled in the related art to which the present invention belongs that the number and kind of the siRNAs constituting the liRNA are not limited, and the present invention has the same effects when the present invention is applied to construct a liRNA targeting mRNA sequences of two or more, that is, 3, or 4 or more different genes.

The liRNA according to the present invention may be characterized in that it specifically inhibits expression of target genes and simultaneously induces an immune reaction. According to one exemplary embodiment of the present invention, the liRNA of the present invention was measured to determine whether the liRNA inhibits the specific expression of the target genes. As a result, it was confirmed that the liRNA had an ability to inhibit expression of genes at a level similar to that of the siRNAs, and such a reaction was carried out in a sequence-dependent manner.

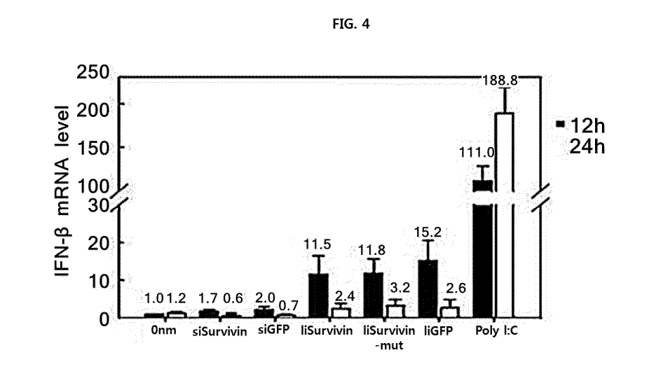

In addition, the immune reaction by the liRNA according to the present invention may be characterized in that it is a reaction by which interferon-β is induced. According to one exemplary embodiment of the present invention, a reaction level of interferon induced by the liRNA according to the present invention was compared with that of liSurvivin-mut. As a result, it was confirmed that the immune reaction occurred in a sequence-independent manner (FIG. 4). Unlike the poly(I:C) in which a level of interferon-β continued to increase after the 24 hour transfection, most of the interferon reactions according to the present invention were knocked down to a baseline level after the 24 hour transfection. Therefore, it was also confirmed that the liRNA of the present invention induced the interferon in a pattern different from that of the poly(I:C).

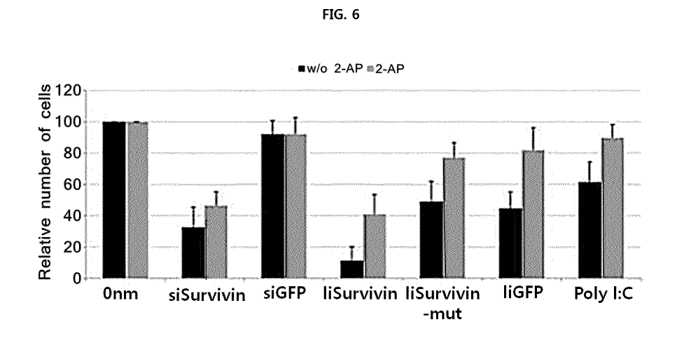

According to another exemplary embodiment of the present invention, PKR dependency of the immune reaction caused by the liRNA was also determined. As a result, it was confirmed that the immune reaction is dependent on the PKR like conventional long dsRNAs such as Poly(I:C) (FIG. 6). Also, when PKR was not activated, that is, liSurvivin cells were treated with a PKR inhibitor, 2-AP, the liRNA showed an ability to inhibit cell growth at a level similar to that of siSurvivin. As a result, it could be seen that the potent anti-proliferative activities of the liRNA according to the present invention was derived from a combination of the PKR-dependent immune reaction and the PKR-independent inhibition of target gene expression.

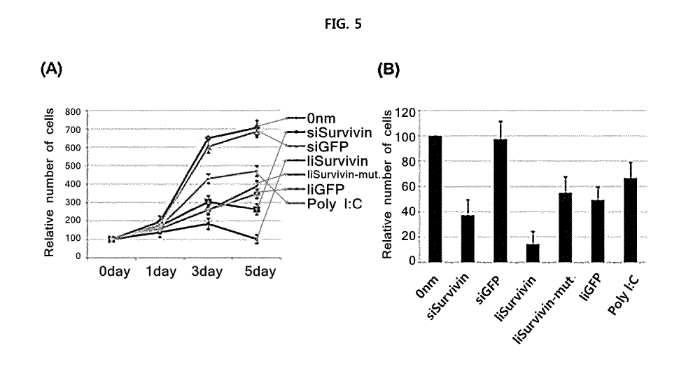

According to one exemplary embodiment of the present invention, the liRNA according to the present invention and conventional siRNAs or non-targetable long dsRNAs were measured for an ability to inhibit growth of cancer cells. As a result, it was confirmed that a combination of the immune reaction and the specific inhibition of gene expression according to the present invention results in a synergistic effect in inhibiting growth of cancer cells, which indicates that the liRNA had an ability to inhibit the growth of cancer cells more effectively than the conventional siRNAs or non-targetable immunostimulatory long dsRNAs (FIG. 5).

Therefore, it was confirmed that the liRNA of the present invention had a synergistic effect (for example, an ability to inhibit growth of cancer cells when the liRNA targets the cancer-associated genes) by specifically inhibiting expression of target genes by means of the siRNA units in the liRNA structure (sequence-dependent) and simultaneously activating PKRs due to structural characteristics of the liRNA to induce interferon-β (structure-dependent but sequence-independent).

According to still another aspect of the present invention, a composition for inhibiting expression of genes or promoting an immune reaction, which includes the liRNA, is provided.

According to still another aspect of the present invention, an antiviral composition including the liRNA in which siRNAs directed against antiviral genes are included as units is provided.

According to yet another aspect of the present invention, an anticancer composition including the liRNA in which siRNAs directed against anticancer genes are included as units is provided.

The composition for inhibiting expression of genes or promoting an immune reaction or the antiviral or anticancer composition according to the present invention may include the liRNA alone or in combination with at least one of a pharmaceutically available carrier, an excipient, and a diluent, and thus may be provided as a pharmaceutical composition. In this case, a pharmaceutically effective amount of the liRNA may be properly included in the pharmaceutical composition according to a disease, severity of the disease, ages, body weight, health condition, and gender of a patient, a route of administration, and treatment time.

As described above, the “pharmaceutically available composition” refers to a composition which is physiologically allowed and does not generally cause allergic reactions or similar reactions such as a gastrointestinal disorder and dizziness when the composition is administered to human beings. Examples of the carrier, the excipient, and the diluent may include lactose, dextrose, sucrose, sorbitol, mannitol, xylitol, erythritol, maltitol, starch, acacia gum, alginate, gelatine, calcium phosphate, calcium silicate, cellulose, methyl cellulose, polyvinylpyrrolidone, water, methyl hydroxybenzoate, propyl hydroxybenzoate, talc, magnesium stearate, and mineral oil, but the present invention is not limited thereto.

### Example 1

Construction of liRNA Structure According to the Present Invention

siRNAs and liRNAs used in this experiment were purchased as RNAs which were chemically synthesized from Bioneer, and prepared through annealing according to the manufacturer's protocol. The liRNAs according to the present invention were prepared by annealing two chemically synthesized 38 nt single-stranded (ss) RNAs (FIG. 1). A 19 nt sequence of the 5′-terminus of an antisense strand is homologous to the corresponding 19 bp sequences of the siRNAs, and a 19 nt overhang complementary to a target mRNA sequence was constructed at the 3′-terminus to ensure annealing with other siRNA units. A 38 nt sense strand was designed to have a 19 nt sequence of the 5′-terminus complementary to the 19 nt sequence of the 5′-terminus of the antisense strand and a 19 nt sequence of the 3′-terminus complementary to the 19 nt sequence of the 3′-terminus of the antisense strand. As a result, long dsRNAs which were multi-ligated through annealing of the two strands to have nicks formed per 19 base pairs were constructed.

To develop an anticancer liRNA, the present inventors constructed a liRNA (liSurvivin) targeting Survivin mRNA. It was revealed that Survivin was an attractive target for anticancer drugs, and inhibition of expression of a Survivin gene using the siRNAs resulted in effective inhibition of cell growth (Chang et al., Mol Ther, 17:725-732, 2009b; Ryan et al., Cancer Treat Rev, 35:553-562, 2009). As a negative control, the present inventors designed liSurvivin in which a seed sequence was engineered (2^{nd} to 7^{th} nucleotides from the 5′-terminus of the antisense sequence were mutated: hereinafter referred to as ‘liSurvivin-mut’) and a liRNA (liGFP) targeting GFP mRNA (FIG. 1).

The sequences and structures of the siRNAs and liRNAs used in this Example are listed in the following Table 1 and shown in FIG. 1.

TABLE 1
Sequences of RNAs used in this study

| siRNA Name | Sequence |
|---|---|
| siSurvivin(AS) | 5′-UGAAAAUGUUGAUCUCCUU(dTdT)-3′ |
| | (SEQ ID NO: 1) |
| siSurvivin(S) | 5′-AAGGAGAUCAACAUUUUCA(dTdT)-3′ |
| | (SEQ ID NO: 2) |
| siGFP(AS) | 5′-UGCGCUCCUGGACGUAGCC(dTdT)-3′ |
| | (SEQ ID NO: 3) |
| siGFP(S) | 5′-GGCUACGUCCAGGAGCGCA(dTdT)-3′ |
| | (SEQ ID NO: 4) |
| liSurvivin(AS) | 5′-UGAAAAUGUUGAUCUCCUUUCCUAAGACAUUGCUAAGG-3′ |
| | (SEQ ID NO: 5) |
| liSurvivin(S) | 5′-AAGGAGAUCAACAUUUUCACCUUAGCAAUGUCUUAGGA-3′ |
| | (SEQ ID NO: 6) |
| lisurvivin- | 5′-UCUUUUAGUUGAUCUCCUUUCCUAAGACAUUGCUAAGG-3′ |
| mut.(AS) | (SEQ ID NO: 7) |
| lisurvivin- | 5′-AAGGAGAUCAACUAAAAGACCUUAGCAAUGUCUUAGGA-3′ |
| mut.(S) | (SEQ ID NO: 8) |
| liGFP(AS) | 5′-UGCGCUCCUGGACGUAGCCUUCGGGCAUGGCGGACUUG-3′ |
| | (SEQ ID NO: 9) |
| liGFP(S) | 5′-GGCUACGUCCAGGAGCGCACAAGUCCGCCAUGCCCGAA-3′ |
| | (SEQ ID NO: 10) |

Also, the liRNA according to the present invention was able to be constructed from units composed of siRNAs targeting different target genes. Accordingly, the present inventors designed a liRNA composed of siSurvivin and siβ-catenin, and also constructed the liRNA including linkers formed between the siRNAs (FIG. 7). The number of the siRNAs constituting the liRNA was not limited, and a liRNA in which two or more, that is, three, or four or more different kinds of siRNAs are repeated was also able to be constructed.

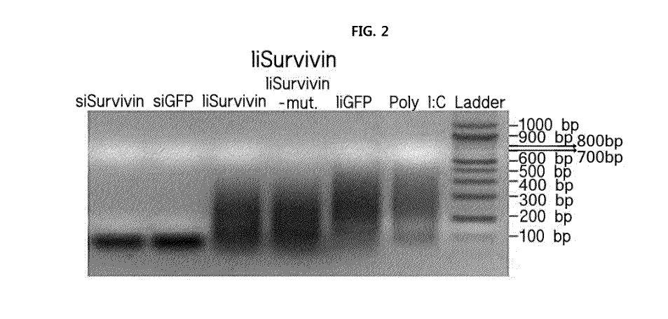

Meanwhile, the size distribution of the liRNAs was analyzed on an agarose gel, and the size distribution results were compared with those of the poly(I:C). It was revealed that the lengths of the liRNAs were in a range of 600 bp or more, a size distribution pattern of which was similar to that of the poly(I:C) (FIG. 2).

### Example 2

### Specific Inhibition of Expression of Target Genes by liRNA

To determine whether the liRNAs according to the present invention induced specific inhibition of target genes, first, HeLa cells were cultured in a Dulbecco-modified Eagle's medium (Gibco) supplemented with 10% fetal bovine serum (FBS), and grown in an antibiotic-free complete medium until cell confluency reached 70%. Before 24 hours of tranfection, the cells were plated on a 12-well plate. The HeLa cells were transfected with siRNA (0.3 nM) or liRNA (0.3 nM) using Lipofectamine 2000 (Invitrogen) according to the manufacturer's protocol.

Next, a total of RNAs were extracted from a cell lysate using an Isol-RNA Lysis Reagent kit (5Prime). Then, the extracted RNAs were used as a template for synthesis of cDNA, and an ImProm-II™ Reverse Transcription System (Promega) were run according to the manufacturer's protocol. Expression levels of mRNAs of Survivin and GAPDH (internal control) were analyzed through qRT-PCR using a step-one real-time PCR system (Applied Biosystems) according to the manufacturer's protocol. Primer sequences of each gene are as described below:

| | | |
|---|---|---|
| GAPDH-forward | 5′-GAG TCA ACG GAT TTG GTC GT-3′ | (SEQ ID NO: 11) |
| GAPDH-reverse | 5′-GAC AAG CTT CCC GTT CTC AG-3′ | (SEQ ID NO: 12) |
| Survivin-forward | 5′-GCA CCA CTT CCA GGG TTT AT-3′ | (SEQ ID NO: 13) |
| Survivin-reverse | 5′-CTC TGG TGC CAC TTT CAA GA-3′ | (SEQ ID NO: 14) |
| IFN-β-forward | 5′-AGA AGT CTG CAC CTG AAA AGA TAT T-3′ | (SEQ ID NO: 15) |
| IFN-β-reverse | 5′-TGT ACT CCT TGG CCT TCA GGT AA-3′ | (SEQ ID NO: 16) |

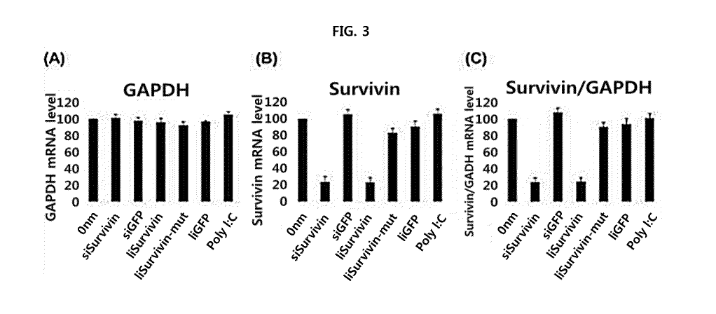

As a result, it was revealed that the liSurvivin effectively knocked down an mRNA level of Survivin to a level similar to that of the siSurvivin, but did not knock down an mRNA level of GAPDH, as shown in FIG. 3. On the other hand, it was revealed that the seed-changed liSurvivin and liGFP did not effectively knock down an mRNA level of Survivin. From these results, it was revealed that the liRNA, that is, a long dsRNA structure having nicks formed therein induced seed sequence-dependent, specific inhibition of expression of target genes. From the facts that the seed-changed liRNA (liSurvivin-mut) did not normally induced inhibition of expression of the target genes, it should also be seen that the specific inhibition of gene expression by the liRNA occurred through an RNAi mechanism.

### Example 3

### Induction of Interferon by liRNAs

Next, the present inventors conducted an experiment on an ability of the liRNAs to induce an interferon reaction in cancer cells. First, HeLa cells were transfected with liSurvivin, liSurvivin-mut, and liGFP, and an expression level of interferon (IFN)-β was measured. Also, the present inventors transfected HeLa cells with siSurvivin, siGFP, and poly(I:C) so as to compare an IFN-β induction level with that of the liRNAs.

As a result, the siRNAs did not induce any interferon reaction in the HeLa cells, as reported previously (Chang et al., Mol. Cells, 27:689-695, 2009a) (FIG. 4). On the other hand, the poly(I:C) induced a strong interferon reaction (>100 fold) after 12 hours and 24 hours of the transfection. Unlike these RNAs, the liRNAs induced a mild level of IFN-β induction reaction (approximately 11 to 16 fold) after 12 hours of the transfection. A level of IFN-β mRNA was mostly knocked down to a baseline level after 24 hours of the transfection, but a level of poly(I:C)-induced IFN-β continued to increase (FIG. 4). These results showed that the transfected liRNAs were able to induce IFN-β expression in different patterns, compared with the poly(I:C).

### Example 4

### Anticancer Activities of PKR-Dependent liRNAs

To determine whether inhibition of growth of cancer cells caused by the liRNAs according to the present invention was carried out in a protein kinase R (PKR)-dependent manner like conventional long dsRNAs, the present inventors confirmed an effect of the liRNAs on inhibition of liRNA-mediated cell growth by pre-treating cells with 5 mM of a PKR inhibitor, 2-AP (Sigma aldrich), before RNA transfection, culturing the cells for 5 days while replacing the used medium with a fresh 5 mM 2-AP-containing medium every 24 hours, and counting the cells.

As a result, treatment of the HeLa cells with 2-AP did not had an effect on inhibition of cell growth by the siSurvivin (FIG. 6). On the other hand, inhibition of cell growth by the liGFP and the seed-changed liSurvivin significantly decreased like the poly(I:C) when the HeLa cells were treated with 2-AP. The treatment with 2-AP resulted in a decrease in inhibition of liSurvivin-mediated cell growth. The liSurvivin showed an ability to inhibit the cell growth similar to that of the siSurvivin when the HeLa cells were treated with 2-AP. This was correspondent to a mechanism in which the sequence-independent antitumor activities of the liRNA structure was dependent on PKR, and the reinforced anti-proliferative activities of the liSurvivin was derived from a combination of PKR-dependent immunostimulation and PKR-independent inhibition of expression of target genes.

### Example 5

### Comparison of Ability of Survivin-Targeted liRNAs and siRNAs or Non-Targetable Long dsRNAs to Inhibit Growth of Cancer Cells

To test whether a combination of immunostimulation and specific inhibition of gene expression caused by oncogene-targeted liRNAs showed reinforced anticancer activities compared with siRNA or immunostimulatory dsRNA alone, the present inventors transfected HeLa cells with liSurvivin, seed-changed liSurvivin, or liGFP, and measured an ability of the liSurvivin, the seed-changed liSurvivin, or the liGFP to inhibit cell growth.

HeLa cells were cultured in a 24-well plate until the cells grew to a density of 2.5×10⁴, and seeded before 24 hours of transfection. Then, a medium was exchanged immediately before the transfection, and 100 n1 of a dilute solution including a transfectant complex in a serum-containing culture medium was added t the cells. All the experiments were performed in duplicate. The cells were stained with trypan blue on days 1, 3, and 5 of treatment, and the viable cells were counted to measure an inhibition level of cell growth.

As a result, the siSurvivin-transfected cells showed decreased cell growth, but the siGFP-transfected cells showed substantially the same cell growth as the control (0 nM), as shown in FIG. 5. The liGFP or liSurvivin-mut showed effective inhibition of cell growth, which was similar to that of the poly(I:C), but showed slightly less inhibition of cell growth than the siSurvivin. These results indicates that, like the poly(I:C), the liRNA structure induces inhibition of growth of cancer cells in a structure-dependent and sequence-independent manner. Among all the tested RNAs, the liSurvivin induced the most potent inhibition of cell growth by completely inhibiting growth of almost all the cancer cells within up to 5 days. From these results, it could be seen that a synergistic effect in inhibiting growth of cancer cells was caused by a combination of Survivin gene knock-down and long dsRNA-mediated immunostimulation.

In the experiments as described above, the present inventors found that the liRNA designed to inhibit expression of Survivin genes had potent anti-proliferative activities against cancer cell lines, and the reinforced anti-proliferative activities of the liRNA were derived from the dual functions of the liRNA structure, that is, i) PKR activities caused due to structural characteristics of the liRNA mimicking the long dsRNAs, and ii) sequence-specific inhibition of expression of oncogenes by siRNA units in the liRNA structure.

Unlike the poly(I:C), the liRNAs of the present invention also induces a mild level of IFN-β, and an induction pattern is not persistent but temporary. One important fact is that this mild level of immunostimulation induces more potent anti-proliferative activities than the poly(I:C) when combined with inhibition of expression of Survivin genes. From these result, it is noted that the liRNA structure according to the present invention may be used as a substitute for the poly(I:C) to develop a dsRNA-based anticancer drug in the near future.

The liRNA according to the present invention includes an ability of the siRNAs constituting the liRNA to inhibit expression of target genes in a sequence-specific manner, and an effect of the liRNA according to the present invention to promote an immune reaction in a structure-dependent manner. For example, when the siRNAs are used as siRNAs targeting cancer-associated genes such as siSurvivin or siβ-catenin, an immune reaction caused by induction of interferon can be promoted together with inhibition of expression of the cancer-associated genes, thereby providing a synergistic effect in inhibiting growth of cancer cells. Accordingly, the liRNA according to the present invention can be very effectively used as an anticancer drug in the near future.

### INDUSTRIAL APPLICABILITY

The siRNAs can be used as siRNAs targeting cancer-associated genes such as siSurvivin or siβ-catenin to inhibit expression of the cancer-associated genes and promote an immune reaction through induction of interferon. Ultimately, the siRNAs can be very useful as an anticancer drug in the near future since the siRNAs have a synergistic effect in inhibiting growth of cancer cells.

It will be apparent to those skilled in the art that various modifications can be made to the above-described exemplary embodiments of the present invention without departing from the scope of the invention. Thus, it is intended that the present invention covers all such modifications provided they come within the scope of the appended claims and their equivalents.

1. A long interfering dsRNA (liRNA) to which a double-stranded siRNA is linearly ligated by means of complementary base-pair binding,
wherein the double-stranded siRNA is composed of an antisense strand and a sense strand, each of which is 19 to 59 nucleotides (nts) in length, wherein the antisense strand and the sense strand are ligated to form a 13 to 50 base pairs (bp) complementary double helix structure, and the double-stranded siRNA has overhangs of 4 to 46 nts in length bound to both the 5′ termini or both the 3′ termini of the double helix structure.

2. The liRNA of claim 1, wherein the overhangs positioned at both the 5′ termini or both the 3′ termini of the double helix structure have sequences complementary to each other, and a melting temperature (Tₘ) of greater than 30° C.

3. The liRNA of claim 1, wherein the antisense strand complementary to the sense strand has a sequence having a homology of at least 70% with respect to an mRNA sequence of a target gene.

4. The liRNA of claim 1, wherein the overhang of the antisense strand has a sequence having a homology of at least 70% with respect to an mRNA sequence of a target gene.

5. The liRNA of claim 1, wherein the antisense strand has a sequence having a homology of at least 70% with respect to mRNA sequences of two or more target genes.

6. The liRNA of claim 1, which has nicks formed per 13 to 50 bp.

7. The liRNA of claim 1, wherein the liRNA targets a cancer-associated gene.

8. The liRNA of claim 7, wherein the cancer-associated gene is Survivin or β-catenin.

9. The liRNA of claim 1, wherein the liRNA specifically inhibits expression of target genes and simultaneously induces an immune reaction.

10. The liRNA of claim 9, wherein the immune reaction is a reaction inducing expression of interferon-β.

11. The liRNA of claim 9, wherein the immune reaction is carried out in a protein kinase R (PKR)-dependent manner.

12. The liRNA of claim 9, wherein the inhibition of the expression of the target genes is carried out in a sequence-dependent manner, and
the immune reaction is carried out in a structure-dependent manner.

13. A composition for inhibiting expression of genes or promoting an immune reaction, which comprises the liRNA of claim 1.

14. An antiviral composition comprising the liRNA of claim 1.

15. An anticancer composition comprising the liRNA of claim 7.


SEQUENCE LISTING (26 sequences)

SEQ ID NO: 1 - DNA, 21 bp, Artificial Sequence
  Description of Artificial Sequence Synthetic antisense strand oligonucleotide of siRNA for Survivin mRNA
ugaaaauguugaucuccuutt

SEQ ID NO: 2 - DNA, 21 bp, Artificial Sequence
  Description of Artificial Sequence Synthetic sense strand oligonucleotide of siRNA for Survivin mRNA
aaggagaucaacauuuucatt

SEQ ID NO: 3 - DNA, 21 bp, Artificial Sequence
  Description of Artificial Sequence Synthetic antisense strand oligonucleotide of siRNA for GFP mRNA
ugcgcuccuggacguagcctt

SEQ ID NO: 4 - DNA, 21 bp, Artificial Sequence
  Description of Artificial Sequence Synthetic sense strand oligonucleotide of siRNA for GFP mRNA
ggcuacguccaggagcgcatt

SEQ ID NO: 5 - RNA, 38 nt, Artificial Sequence
  Description of Artificial Sequence Synthetic antisense strand oligonucleotide of liSurvivin
ugaaaauguugaucuccuuuccuaagacauugcuaagg

SEQ ID NO: 6 - RNA, 38 nt, Artificial Sequence
  Description of Artificial Sequence Synthetic sense strand oligonucleotide of liSurvivin
aaggagaucaacauuuucaccuuagcaaugucuuagga

SEQ ID NO: 7 - RNA, 38 nt, Artificial Sequence
  Description of Artificial Sequence Synthetic antisense strand oligonucleotide of mutant-liSurvivin
ucuuuuaguugaucuccuuuccuaagacauugcuaagg

SEQ ID NO: 8 - RNA, 38 nt, Artificial Sequence
  Description of Artificial Sequence Synthetic sense strand oligonucleotide of mutant-liSurvivin
aaggagaucaacuaaaagaccuuagcaaugucuuagga

SEQ ID NO: 9 - RNA, 38 nt, Artificial Sequence
  Description of Artificial Sequence Synthetic antisense strand oligonucleotide of liGFP
ugcgcuccuggacguagccuucgggcauggcggacuug

SEQ ID NO: 10 - RNA, 38 nt, Artificial Sequence
  Description of Artificial Sequence Synthetic sense strand oligonucleotide of liGFP
ggcuacguccaggagcgcacaaguccgccaugcccgaa

SEQ ID NO: 11 - DNA, 20 bp, Artificial Sequence
  Description of Artificial Sequence Synthetic GAPDH-forward primer
gagtcaacggatttggtcgt

SEQ ID NO: 12 - DNA, 20 bp, Artificial Sequence
  Description of Artificial Sequence Synthetic GAPDH-reverse primer
gacaagcttcccgttctcag

SEQ ID NO: 13 - DNA, 20 bp, Artificial Sequence
  Description of Artificial Sequence Synthetic Survivin-forward primer
gcaccacttccagggtttat

SEQ ID NO: 14 - DNA, 20 bp, Artificial Sequence
  Description of Artificial Sequence Synthetic Survivin-reverse primer
ctctggtgccactttcaaga

SEQ ID NO: 15 - DNA, 25 bp, Artificial Sequence
  Description of Artificial Sequence Synthetic IFN-beta-forward primer
agaagtctgcacctgaaaagatatt

SEQ ID NO: 16 - DNA, 23 bp, Artificial Sequence
  Description of Artificial Sequence Synthetic IFN-beta-reverse primer
tgtactccttggccttcaggtaa

SEQ ID NO: 17 - RNA, 76 nt, Artificial Sequence
  Description of Artificial Sequence Synthetic liRNA oligonucleotide
ugaaaauguugaucuccuuuccuaagacauugcuaaggugaaaauguugaucuccuuuccuaagacauugcuaagg

SEQ ID NO: 18 - RNA, 76 nt, Artificial Sequence
  Description of Artificial Sequence Synthetic liRNA oligonucleotide
aaggagaucaacauuuucaccuuagcaaugucuuaggaaaggagaucaacauuuucaccuuagcaaugucuuagga

SEQ ID NO: 19 - RNA, 76 nt, Artificial Sequence
  Description of Artificial Sequence Synthetic liRNA oligonucleotide
ucuuuuaguugaucuccuuuccuaagacauugcuaaggucuuuuaguugaucuccuuuccuaagacauugcuaagg

SEQ ID NO: 20 - RNA, 76 nt, Artificial Sequence
  Description of Artificial Sequence Synthetic liRNA oligonucleotide
aaggagaucaacuaaaagaccuuagcaaugucuuaggaaaggagaucaacuaaaagaccuuagcaaugucuuagga

SEQ ID NO: 21 - RNA, 76 nt, Artificial Sequence
  Description of Artificial Sequence Synthetic liRNA oligonucleotide
ugcgcuccuggacguagccuucgggcauggcggacuugugcgcuccuggacguagccuucgggcauggcggacuug

SEQ ID NO: 22 - RNA, 76 nt, Artificial Sequence
  Description of Artificial Sequence Synthetic liRNA oligonucleotide
ggcuacguccaggagcgcacaaguccgccaugcccgaaggcuacguccaggagcgcacaaguccgccaugcccgaa

SEQ ID NO: 23 - RNA, 76 nt, Artificial Sequence
  Description of Artificial Sequence Synthetic liRNA oligonucleotide
ugaaaauguugaucuccuuguccaucaauaucagcuacugaaaauguugaucuccuuguccaucaauaucagcuac

SEQ ID NO: 24 - RNA, 76 nt, Artificial Sequence
  Description of Artificial Sequence Synthetic liRNA oligonucleotide
aaggagaucaacauuuucaguagcugauauugauggacaaggagaucaacauuuucaguagcugauauugauggac

SEQ ID NO: 25 - RNA, 58 nt, Artificial Sequence
  Description of Artificial Sequence Synthetic liRNA oligonucleotide
ugaaaauguugaucuccuugcgcgcgcgcguccaucaauaucagcuacgcgcgcgcgc

SEQ ID NO: 26 - RNA, 58 nt, Artificial Sequence
  Description of Artificial Sequence Synthetic liRNA oligonucleotide
aaggagaucaacauuuucagcgcgcgcgcguagcugauauugauggacgcgcgcgcgc